(19) United States
(12) Patent Application Publication
(10) Pub. No.: US 2010/0274040 A1
(43) Pub. Date: Oct. 28, 2010

(54) 1,1'-Bi-2-Naphthol Derivatives Having (Meth)acrylate Groups

(76) Inventors: Cheng Chao PAI, Tayuan Hsiang (TW); Hsiu Chi Lai, Tayuan Hsiang (TW); Ching Ying Wu, Tayuan Hsiang (TW); Kun Lin Lee, Tayuan Hsiang (TW)

Correspondence Address: Muncy, Geissler, Olds & Lowe, PLLC, 4000 Legato Road, Suite 310, FAIRFAX, VA, 22033, US

(21) Appl. No.: 12/499,563

(22) Filed: Jul. 8, 2009

(30) Foreign Application Priority Data
Apr. 24, 2009 (TW) 098113674

(51) Int. Cl. C07C 69/76

(52) U.S. Cl. 560/80

(57) ABSTRACT

The present invention relates to 1,1′-bi-2-naphthol derivatives having (meth)acrylate groups, which can be used for preparing photocurable coating and has a refractive index of 1.53 or more. By using the present 1,1′-bi-2-naphthol derivatives, optical components with high refractive index and less light dispersion can be produced.

### FIELD OF THE INVENTION

The present invention relates to novel 1,1-bi-2-naphthol derivatives having (meth)acrylate groups which exhibit low viscosity, low skin irritative and have high refractive index and are easily produced.

### BACKGROUND OF THE INVENTION

Introducing of functional group and blending inorganic nanometer material into polymers are usually employed to adjust the refractive index of the polymer to increase its refractive index. The method for introducing functional group to increase the refractive index of polymer can be classified into the follows: (1) A method for introducing an aromatic group or a fused ring into polymer structure to increase the refractive index of the polymer. However, introducing of the aromatic group or the fused ring will result in the polymer possessing higher chromatic dispersion and lower Abbe number. (2) A method for introducing halogen atoms except fluorine atom into polymer structure to increase the refractive index of the polymer. However, introducing of halogen atoms will increase the density of the resultant polymer and results in poor weather resistance and yellowing. Also, use of halogen is not allowed in environment protection act. (3) A method for introducing heavy metal ion such as lead, lanthanum, and niobium into polymer to increase the refractive index. However, introducing of heavy metal ion will increase the density of the resultant polymer and lower impact resistance and is easily yellowing so that it is unpractical. (4) A method for introducing aliphatic polycyclic group into polymer to increase the refractive index and lower chromatic dispersion. (5) A method for introducing heteroatoms such as sulfur, nitrogen, or phosphorous atom into polymer structure to increase the refractive index. However, its reaction conditions are complicated so that it is unpractical in industrial production. (6) A method for blending inorganic nanometer material into polymer to increase the refractive index. However, the resultant polymer has inferior transparency. Among the above methods, the method for introducing sulfur atom into polymer structure is the most effective to increase the refractive index meanwhile the resultant polymer exhibits less chromatic dispersion, but it also possesses drawbacks of remaining thiol odor and insufficient stability so that its production operation is not acceptable.

There are many reports regarding the methods introducing sulfur atom into polymer structure to increase refractive index recently, which usually use a monomer having (meth)acrylate group that is obtained from introducing sulfur-containing functional group such as thioether group, thioester, thiocarbamate group, and sulfone group into sulfur-containing hetercyclic ring and reacting the sulfur-containing functional group with (meth)acrylate, as disclosed in U.S. Pat. Nos. 6,835,844; 6,541,591; 6,265,510; 5,183,917. According to the above methods, since a sulfur-containing functional group is introduced into the monomer, the sulfur content of the monomer is relative high, for example, more than 50%. Although it effectively increases the refractive index of the polymer, the Abbe number of the polymer is also higher.

High refractive index for the material used in optical application fields such as lens, prism, optical coating, displayer, brightening film used in light emitting diode (LED), and a binder used in packaging is necessary. Most high refractive index resins are thermal-set resin, which has been developed rapidly and its refractive index can be increased by various methods. The method for increasing refractive index by changing electrons in original molecular such as introducing benzene ring or halogen or sulfur atoms has been disclosed in the above-mentioned US patents.

Moreover, in U.S. Pat. No. 7,446,159 and U.S. Pat. No. 7,297,810, they disclosed a resin composition having an appropriate viscosity and capable of using as packaging resin for LED and optical brightening film. The main component in the resin composition is bifunctional (meth)acrylate or monofunctional (meth)acrylate monomer having a fluorene group as the hardening resin. It is also found that the hardening article formed from the resin composition possesses a refractive index of 1.55 or more, which is not less than that of epoxy resin. Moreover, the hardening article possesses excellent heat resistance and light resistance and exhibits hardness suitable for using as packaging resin. It is preferable that the monomer contained in the resin composition does not contain sulfur-containing monomer since the sulfur-containing monomer will result in yellowing problem. For the purpose of increasing refractive index of the hardening article, a method of adding super fine metal oxide particles such as titanium dioxide, zirconium oxide, zinc oxide, aluminum oxide or magnesium oxide into resin compositions is disclosed.

(Meth)acrylate having 1,1′-bis-2-naphthol structure has been disclosed in reference such as Polymer Preprints (American Chemical Society, Division of Polymer Chemistry; 2001, 42(1), 452-453). However, only methacrylate-1,1′-bis-2-naphthol and (ethyleneoxy)₁-metharcylate-1,1′-bis-2-naphthol have been disclosed.

In U.S. Pat. No. 7,413,782; U.S. Pat. No. 7,425,356, US patent publication No. 2008/0272337 and GB No. 2298202 and reference such as POLYMER ENGINEERING AND SCIENCE, 1997, Vol. 37, NO. 6, P945, they mention that if bisnaphthol possesses pure chirality, it can produce chiral film or coating for reflecting polarizing light in one direction. The above compounds preferably exhibit good solubility in liquid crystal mixture so that exhibit a broader liquid crystal phase. By changing the substituents on the naphthalene ring, a bisnaphthol having properties of high twisting power (HTP), easily polymerizing, easily orientation, and easily coating can be obtained. However, the process for preparing such bisnaphthol derivatives requires many synthesis steps and is complicated.

Furthermore, JPA 2008-247755 disclosed a linear bi-o-phenylphenol acrylate, which is a curable monomer exhibiting high heat resistance and high refractive index, and can be used as skin-forming material, solder resist for producing printed circuit board, anti-erosive agent for electroplating, optical fiber, light waveguide, and so on. However, the starting material for synthesizing the acrylate is not easily obtained.

### SUMMARY OF THE INVENTION

The purpose of the present invention is to provide 1,1′-bi-2-naphthol derivatives having (meth)acrylate groups, which can be synthesized easily and simply, can be produced in industrial scale, and is suitable for using in optical elements.

The present invention relates to 1,1′-bi-2-naphthol derivatives having (meth)acrylate groups, which can be used as a monomer for preparing an optical polymer resin and a photo-curable coating composition. An article cured from the polymer resin and photo-curable coating composition prepared from the present 1,1′-bi-2-naphthol derivatives having (meth)acrylate groups exhibits a refractive index of not less than 1.60. When applying in adhesive or polymer coating fields, it usually select the adhesive and polymer coating which refractive index is equal to or higher than that of the substrate to be used, for example, the refractive index of polymethyl methacrylate (PMMA) is 1.489; the refractive index of polycarbonate (PC) is 1.585; the refractive index of polyethylene terephthalate (PET) is 1.64. By selecting the adhesive and polymer coating which refractive index is equal to or higher than that of the substrate to be used, the problems of color difference and chromatic dispersion due to the large refractive index difference between them can be avoided.

In summary, the present invention relates to a 1,1′-bi-2-naphthol derivative having (meth)acrylate groups, which is presented by the following formula (I):

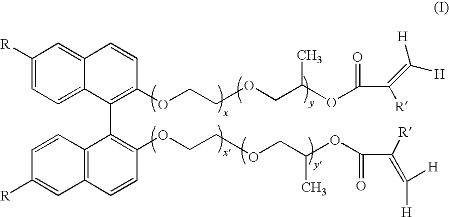

wherein the C₂-axial is in S-configuration, R-configuration, or racemic;
   R's are the same or different, and each represents a hydrogen atom, a halogen atom except fluorine atom, —NO₂, —NCO or NR₁R₂, wherein R₁ and R₂ are the same or different, and each represents a hydrogen atom, a straight, branched, or cyclic alkyl group having 1 to 20 carbon atoms, or an aryl group having 6 to 20 carbon atoms;
R′ represents a hydrogen atom or a methyl group; and
x+x′+y+y′ is an integer of from 3 to 30.

The 1,1′-bi-2-naphthol derivative having (meth)acrylate groups of the present invention is prepared by using 1,1′-bi-2-naphthol (brief referred as BINOL) as a starting material, the 1,1′-bi-2-naphthol can be produced by oxidative coupling 2-naphthol and its derivatives.

Optical active BINOLs (either in R- or S-configuration) are bis-aromatic compound typically having asymmetrical C₂ axial and possess unique stereo-chemical properties and are easily isolated (Organic Synthesis Coll. Vol. 10, 2004, P93; Vol. 76, 1999, P1) or can be directly unsymmetrical catalytic oxidative synthesized into an enantiomer in high purity (Organic Synthesis Coll. Vol. 9, 1998, P77; Vol. 70, 1992, P60), and can be used for identifying molecular and synthesizing new material in unsymmetrical reaction. The optical active BINOLs are represented by the following formulae:

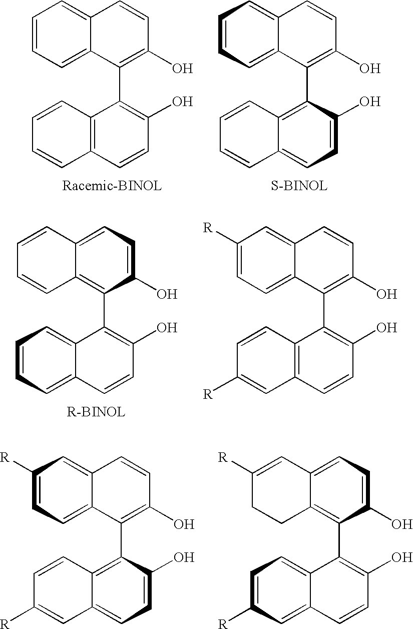

wherein, each R's is the same or different and represents a hydrogen atom, a halogen atom except fluorine atom, —NO₂, —NCO or —NR₁R₂. By halogenating (such as chlorinating, brominating, or iodinating) BINOL in R—, S—, or racemic configuration and then nitrating of the resultant halogenated BINOL with fume nitric acid, it will result in 6,6′-di-nitrated BINOL. The resultant 6,6′-di-nitrated BINOL can be further reduced to obtain 6,6′-diamino BINOL, which may be converted into secondary amine, tertiary amine. The 6,6′-diamino BINOL can further be reacted with bisphosgene to obtain high reactive isocyanine.

The present invention is directed to development of a 1,1′-bi-2-naphthol derivative having (meth)acrylate groups which can be used to prepare polymer having high refractive index and is applicable in optical material.

The 1,1′-bi-2-naphthol derivative having (meth)acrylate groups according to the present invention is prepared by first additional reacting ethylene oxide (EO) and/or propylene oxide (PO) to BINOL to obtain an intermediate of the following formula (II):

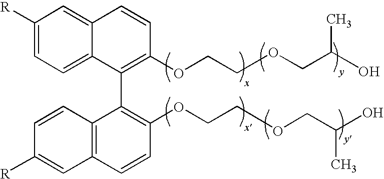

wherein the C₂-axial is in S-configuration, R-configuration, or racemic;
   R's are the same or different, and each represents a hydrogen atom, a halogen atom except fluorine atom, —NO₂, —NCO or NR₁R₂, wherein R₁ and R₂ are the same or different, and each represents a hydrogen atom, a straight, branched, or cyclic alkyl group having 1 to 20 carbon atoms, or an aryl group having 6 to 20 carbon atoms; and
x+x′+y+y′ is an integer of from 3 to 30.

The addition reaction between BINOL and ethylene oxide (EO) and/or propylene oxide (PO) is carried out in a basic condition, for example, in the presence of basic compound such as NaOH, KOH, NaH, KH in an autoclave in suitable solvent and in the presence of phase-transfer catalyst with purging ethylene oxide (EO) and/or propylene oxide (PO) gases. The ethylene oxide (EO) and propylene oxide (PO) can be used alone or as a mixture thereof. The EO and PO units can be arranged in random or block after addition reaction with BINOL.

The above addition reaction is carried out at a temperature of from room temperature to 200° C., preferable from 80˜150° C., at a pressure of up to 2000 psi, preferable at 100 psi. The additional reaction is carried out in inert solvent such as toluene, xylene, and mesitylene, dimethylformamide (DMF), dimethylsulfoxide (DMSO), and tetrahydrofuran (THF). The phase transfer catalyst can use, for example, quaternary ammonium salts in an amount of catalytic amount, and preferably use in an amount of from 0.01 to 3.0 wt %, preferably 0.1 wt % based on the total weight of the reaction mixture. After completing the reaction, a suitable acid, such as phosphoric acid or acetic acid, is used to neutralize the base remained in the reaction mixture. In the reaction, an antioxidant, for example, a phenol type antioxidant and phosphorous-containing antioxidant can be added in an appropriate amount to prevent coloring of the product due to high temperature reaction. Also, the addition number of EO and/or PO is preferable in the range of from 3 to 30, respectively.

Then, the intermediate of formula (II) is reacted with (meth)acrylic acid to subject to esterification to produce 1,1′-bi-2-naphthol derivative having (meth)acrylate groups of the present invention. The esterification is conducted at a normal pressure or a reduced pressure while azeotropic distillation is carried out with benzenes or alkanes to remove water generated in the esterification. It is preferable to add suitable free radical inhibitor into the reaction to prevent gelling. The esterification is preferably conducted in acidic condition, for example, in the presence of acid such as methane sulfonic acid, p-toluene sulfonic acid and the like. The acid is preferably selected to be those capable of reacting at lower temperature (below 100° C.). Examples of benzenes solvents include, for example, benzene, toluene, and xylene and the like. Examples of alkanes solvents include, for example, hexane and heptane and the like. The free radical inhibitor can be, for example, hydroquinone (HQ), hydroquinone monomethyl ether (HQME), nitrobenzene, and butyl hydroxyl toluene (BHT) and oxygen contained in the air.

The process for preparing 1,1′-bi-2-naphthol derivative having (meth)acrylate groups of the present invention is simple and when the 1,1′-bi-2-naphthol derivative having (meth)acrylate groups is used to prepare photo-curable coating or photo-sensitive resin composition for optical elements, its hardening article exhibits high refractive index at least 1.60.

The 1,1′-bi-2-naphthol derivative having (meth)acrylate groups of the present invention is a monomer exhibiting high refractive index, the starting material for preparing the same is easily obtainable, its preparation is simple, and there is no remaining odor during operation. The 1,1′-bi-2-naphthol derivative having (meth)acrylate groups of the present invention monomer are suitable for using in preparing optical elements, LED package, liquid crystal component, brightening film, ophthalmic lens, and optical plastic camera lens, and the like.

### DETAILED DESCRIPTION OF THE INVENTION

### EXAMPLE 1

### Synthesis of (ethyleneoxy)₃-1,1′-bi-2-naphthol-bisacrylate

Into one-liter flask equipped with an air inlet, a mechanical stirrer, a water separating line, a condenser, and a heating apparatus, were added with 209 g of (ethyleneoxy)₃-1,1-bi-2-naphthol (the above formula (II) wherein the sum of x and x′ is equal to 3, and both y and y′ are zero), 79.2 g of acrylic acid, 5.48 g of methane sulfonic acid, 360 g of toluene as solvent, 0.32 g of hydroquinone monomethyl ether, and 0.1 6 g of nitrobenzene. The mixture was heated to reflux for 9 hours and the generated water was collected to determine whether the reaction completed. After completing the reaction, the reaction mixture was cooled to room temperature and the organic layer was washed and neutralized with an appropriate amount of aqueous sodium hydroxide to remove the acid present in the mixture and the aqueous layer was discarded, and the washing procedure was repeated twice again to remove the aqueous layer. The result organic layer was condensed under reduced pressure and dried in vacuum at a temperature of from 90˜92° C. to remove the solvent and water and finally filtered to obtain the subject compound, yield: 79.52%. The physiochemical properties of the resultant compound were analyzed by the following methods.
Testing of refractive index: The refractive index was measured at 25° C. by using ATAGO Refractometer RX-5000 instrument a.
Viscosity test: The viscosity was measured at 25° C. by using Brookfield
DV-E Viscometer, #4 spindle.
The results are as follows.
Appearance: yellow liquid (observed with the naked eye)
Visicosity (#4; 25° C.): 75000 cps
Refractive index (n²⁵_{d}): 1.54661

### EXAMPLE 2

### Synthesis of (ethyleneoxy)₆-1,1′-bi-2-naphthol-bisacrylate

Into one-liter flask equipped with an air inlet, a mechanical stirrer, a water separating line, a condenser, and a heating apparatus, were added with 350 g of (ethyleneoxy)₆-1,1-bi-2-naphthol (the above formula (II) wherein the sum of x and x′ is equal to 6, and both y and y′ are zero), 100.8 g of acrylic acid, 6.98 g of methane sulfonic acid, 300 g of toluene as solvent, 0.379 g of hydroquinone monomethyl ether, and 0.1909 g of nitrobenzene. The mixture was heated to reflux for 9 hours and the generated water was collected to determine whether the reaction completed. After completing the reaction, the reaction mixture was cooled to room temperature and the organic layer was washed and neutralized with an appropriate amount of aqueous sodium hydroxide to remove the acid present in the mixture and the aqueous layer was discarded, and the washing procedure was repeated twice again to remove the aqueous layer. The result organic layer was condensed under reduced pressure and dried in vacuum at a temperature of from 90˜92° C. to remove the solvent and water and finally filtered to obtain the subject compound, yield: 86.54%. The physiochemical properties of the resultant compound were as follows.
Appearance: yellow liquid (observed with the naked eye)
Visicosity (#4; 25° C.): 4520 cps
Refractive index (n²⁵_{d}): 1.54128

### EXAMPLE 3

### Synthesis of (propyleneoxy)₆-1,1′-bi-2-naphthol-bisacrylate

Into one-liter flask equipped with an air inlet, a mechanical stirrer, a water separating line, a condenser, and a heating apparatus, were added with 317 g of (propyleneoxy)₆-1,1-bi-2-naphthol (the above formula (II) wherein the sum of y and y′ is equal to 6, and both x and x′ are zero), 79.2 g of acrylic acid, 8.23 g of methane sulfonic acid, 360 g of toluene as solvent, 0.377 g of hydroquinone monomethyl ether, and 0.189 g of nitrobenzene. The mixture was heated to reflux for 12 hours and the generated water was collected to determine whether the reaction completed. After completing the reaction, the reaction mixture was cooled to room temperature and the organic layer was washed and neutralized with an appropriate amount of aqueous sodium hydroxide to remove the acid present in the mixture and the aqueous layer was discarded, and the washing procedure was repeated once again to remove the aqueous layer. The result organic layer was condensed under reduced pressure and dried in vacuum at a temperature of from 90˜92° C. to remove the solvent and water and finally filtered to obtain the subject compound, yield: 81.04%. The physiochemical properties of the resultant compound were as follows.
Appearance: yellow liquid (observed with the naked eye)
Visicosity (#4; 25° C.): 18160 cps
Refractive index (n²⁵_{d}): 1.55633

### EXAMPLE 4

### Synthesis of (ethyleneoxy)₁₀-1,1′-bi-2-naphthol-bisacrylate

Into one-liter flask equipped with an air inlet, a mechanical stirrer, a water separating line, a condenser, and a heating apparatus, were added with 363 g of (ethyleneoxy)₁₀-1,1-bi-2-naphthol (the above formula (II) wherein the sum of x and x′ is equal to 10, and both y and y′ are zero), 79.2 g of acrylic acid, 8.23 g of methane sulfonic acid, 360 g of toluene as solvent, 0.377 g of hydroquinone monomethyl ether, and 0.189 g of nitrobenzene. The mixture was heated to reflux for 10 hours and the generated water was collected to determine whether the reaction completed. After completing the reaction, the reaction mixture was cooled to room temperature and the organic layer was washed and neutralized with an appropriate amount of aqueous sodium hydroxide to remove the acid present in the mixture and the aqueous layer was discarded, and the washing procedure was repeated once again to remove the aqueous layer. The result organic layer was condensed under reduced pressure and dried in vacuum at a temperature of from 90˜92° C. to remove the solvent and water and finally filtered to obtain the subject compound, yield: 85.50%. The physiochemical properties of the resultant compound were as follows.
Appearance: yellow liquid (observed with the naked eye)
Visicosity (#4; 25° C.): 1990 cps
Refractive index (n²⁵_{d}): 1.56526

### EXAMPLE 5

### Synthesis of (propyleneoxy)₁₀-1,1′-bi-2-naphthol-bisacrylate

Into one-liter flask equipped with an air inlet, a mechanical stirrer, a water separating line, a condenser, and a heating apparatus, were added with 346.4 g of (propyleneoxy)₁₀-1,1-bi-2-naphthol (the above formula (II) wherein the sum of y and y′ is equal to 10, and both x and x′ are zero), 63.36 g of acrylic acid, 4.39 g of methane sulfonic acid, 340 g of toluene as solvent, 0.357 g of hydroquinone monomethyl ether, and 0.179 g of nitrobenzene. The mixture was heated to reflux for 14 hours and the generated water was collected to determine whether the reaction completed. After completing the reaction, the reaction mixture was cooled to room temperature and the organic layer was washed and neutralized with an appropriate amount of aqueous sodium hydroxide to remove the acid present in the mixture and the aqueous layer was discarded, and the washing procedure was repeated once again to remove the aqueous layer. The result organic layer was condensed under reduced pressure and dried in vacuum at a temperature of from 90˜92° C. to remove the solvent and water and finally filtered to obtain the subject compound, yield: 81.05%. The physiochemical properties of the resultant compound were as follows.
Appearance: yellow liquid (observed with the naked eye)
Visicosity (#4; 25° C.): 3620 cps
Refractive index (n²⁵_{d}): 1.53270

What is claimed is:

1. A 1,1′-bi-2-naphthol derivative having (meth)acrylate groups, which is presented by the following formula (I): 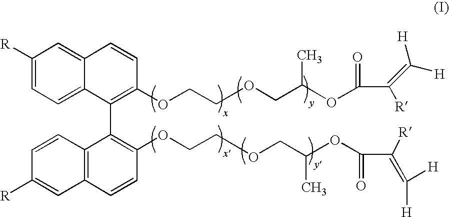
wherein the C₂-axial is in S-configuration, R-configuration, or racemic;
R's are the same or different, and each represents a hydrogen atom, a halogen atom except fluorine atom, —NO₂, —NCO or NR₁R₂, wherein R₁and R₂ are the same or different, and each represents a hydrogen atom, a straight, branched, or cyclic alkyl group having 1 to 20 carbon atoms, or an aryl group having 6 to 20 carbon atoms;
R′ represents a hydrogen atom or a methyl group; and
x+x′+y+y′ is an integer of from 3 to 30.

2. The 1,1′-bi-2-naphthol derivative having (meth)acrylate groups according to claim 1, wherein the sum of x and x′ is an integer of from 3 to 10; and both y and y′ are equal to zero.

3. The 1,1′-bi-2-naphthol derivative having (meth)acrylate groups according to claim 1, wherein the sum of both x and x′ are equal to zero; and the sum of y and y′ is an integer of from 3 to 10.
